(19) United States
(12) Patent Application Publication
(10) Pub. No.: US 2022/0370372 A1
(43) Pub. Date: Nov. 24, 2022

(54) BISPHOSPHONATE LOADED STARCH NANOPARTICLE

(71) Applicant: GreenMark Biomedical Inc., East Lansing, MI (US)

(72) Inventors: Steven BLOEMBERGEN, Okemos, MI (US); Nathan A. JONES, Ann Arbor, MI (US)

(21) Appl. No.: 17/761,057

(22) PCT Filed: Sep. 25, 2020
(86) PCT No.: PCT/US2020/052794

§ 371 Date: Mar. 16, 2022

Related U.S. Application Data
(60) Provisional application No. 62/906,865, filed on Sep. 27, 2019.

(51) Int. Cl. A61K 9/51; A61K 33/42

(52) U.S. Cl. CPC A61K 9/5161; A61K 9/5192; A61K 33/42

(57) ABSTRACT

A phosphorous compound such as STMP is used as a cross-linking agent while making a starch nanoparticle with a bisphosphonate drug in an emulsion process. Negative charge of the nanoparticle is optionally reduced or reversed by adding cations and/or cationizing the starch optionally while forming the nanoparticles. Anionic active agents, such as a bisphosphonate, are optionally incorporated into the nanoparticle during the formation process. For example, a bisphosphonate salt can be added, which promotes the crosslinking reaction while also providing bisphosphonate in the nanoparticle. The retention of both calcium and bisphosphonate in the nanoparticle is improved when both salts are used. Alternatively, the nanoparticle may be used without added calcium. The nanoparticles may be useful for the treatment of osteoporosis or other skeletal disorders or cancer.

### RELATED APPLICATIONS

This application claims priority from, and the benefit of, U.S. provisional patent application No. 62/906,865 filed on Sep. 27, 2019, which is incorporated herein by reference.

### FIELD

This specification relates to biopolymer nanoparticles containing a bisphosphonate compound and to methods of making the nanoparticle. The specification also relates to the treatment of cancer and osteoporosis or other skeletal conditions.

### BACKGROUND

Bisphosphonates, such as Alendronate (alendronate sodium hydrate or alendronic acid), have been proposed for use in treating osteoporosis. The bisphosphonates act on osteoclasts to inhibit bone resorption, thereby increasing bone mineral density. Bisphosphonates, such as Zoledronate (zoledronic acid), have also been used to treat skeletal complications of metastatic breast cancer and prostate cancer.

There is also evidence that bisphosphonates have an anti-tumor effect by way of their effects on tumor associated macrophages (TAMs), as described for example in Rogers and Holen Journal of Translational Medicine 2011, 9:177. However, bisphosphonate drugs delivered intravenously may accumulate in bone tissue rather than around tumors. US Patent Application Publication US 2016/0220692 A1, entitled Targeting the M2-Tumor Associated Macrophage for Cancer Therapy, discloses methods of directly targeting specific cell surface receptors on M2 macrophages for antibody or nanoparticle directed therapy. Examples of bisphosphonate drugs suitable for treating the M2-TAMs are described.

### INTRODUCTION

The following section is intended to introduce the reader to the invention and the detailed description to follow but not to limit or define any claimed invention.

Nanoparticles are capable of both passive and active targeting. Starch based nanoparticles in particular can be made in various sizes, for example from 20 nm to 600 nm, and with positive or negative zeta potential. Starch based nanoparticles can also be attached to targeting compounds (ligands) including, for example, mannose brushes or aptamers, which interact with receptors on the surface of M2-TAMs.

In a process described herein, a phosphorous compound such as STMP is used as a cross-linking agent while making a starch nanoparticle. The cross-linking agent thereby provides crosslinked nanoparticles with a negative charge. However, as described herein, the negative charge can optionally be reduced, neutralized or reversed by adding preferably multi-valent cations and/or cationizing the starch, one or both of which may be done optionally while forming the nanoparticles. The addition of a calcium salt in the process of making the nanoparticle for example serves to make the charge of the nanoparticle partially, nearly or completely neutral. The addition of cations such as calcium also appears to increase the retention of anionic active agents, such as bisphosphonates. A bisphosphonate may be incorporated into the nanoparticle during the formation process, for example by way of adding a bisphosphonate salt. The bisphosphonate is an active agent useful in the treatment of, for example, osteoporosis and/or cancer. The presence of phosphorous and optionally calcium in the nanoparticle may also be beneficial in the treatment of osteoporosis or other skeletal conditions.

This specification describes methods of making starch based nanoparticles made with a phosphate crosslinker and a bisphosphonate according to an emulsion process, and the resulting starch based nanoparticles. Optionally, the nanoparticles have a cation and/or cationic moieties on the starch. The cation and/or cationic moieties may be added while making the nanoparticles. The nanoparticles may be used in one or more methods of therapeutic treatment such as the treatment of osteoporosis, other skeletal conditions, or cancer. Optionally, the nanoparticles may be targeted with a ligand for receptors on TAMs such as M2-TAMs.

In various processes described herein, starch based nanoparticles are made using an emulsion process such as a phase inversion emulsion process. The biopolymer is cross-linked with a phosphate cross-linker, for example STMP, optionally in the presence of alkali and sodium or other salts to increase the ionic strength and catalyze the crosslinking reaction. A bisphospohonate active agent is present in the water phase of a water-in-oil emulsion. Optionally, one or more of (i) a multivalent cation, such as calcium, and (ii) one or more starch cationizing agents, are present in the water phase of the water-in-oil emulsion. Compounds may be added to the water phase while the water phase is emulsified (i.e. after phase inversion), during the phase inversion, or before the water phase is emulsified (i.e. before phase inversion). The water phase also contains the starch and phosphate cross-linker. In some examples, a bisphosphonate salt is added to the water phase before phase inversion. In some examples, a calcium salt and/or one or more starch cationizing agents are added to the water phase during or after phase inversion to produce a further decrease in negative charge, or to produce a net positive charge.

Various nanoparticles described herein comprises starch, phosphorous, bisphosphonate and optionally calcium. The phosphorous may include one or more starch-phosphate compounds and/or dangling phosphates. Optionally, the nanoparticles have a positive zeta potential at a pH of 7.0 or less or a pH of 5.5 or less. Optionally, the nanoparticles may have a negative zeta potential at a pH of 7.0 or more. Optionally, the nanoparticles may have a size in the range of 100-700 nm or 100-500 nm as determined by the peak intensity or Z-average size in dynamic light scattering (DLS) or as determined by the mean size or D50 in nanoparticle tracking analysis (NTA).

This specification also describes the use of nanoparticles to treat a condition such as osteoporosis, cancer, or a skeletal complication of a cancer. The method includes delivering nanoparticles as described herein to a patient. The nanoparticles may be delivered to a patient orally, by injection at a tumor site, or by intravenous injection. The nanoparticles may release a bisphosphonate active agent as the nanoparticles degrade by action of amylase in the mouth, while the nanoparticles are in other parts of the alimentary system, while the nanoparticles are circulating in the bloodstream, while the nanoparticles are associated with bone or a tumor, or after the nanoparticles are taken up into cells.

### BRIEF DESCRIPTION OF FIGURES

FIG. 1 is a schematic process flow diagram of biopolymer nanoparticle formation by way of phase inversion emulsion.

FIG. 2 is a graph of the release of Alendronate from starch nanoparticles over the first 30 minutes after dialysis in PBS.

FIG. 3 is a graph of the release of Alendronate from starch nanoparticles after dialysis in PBS over about 2 weeks.

### DETAILED DESCRIPTION

Without intending to be limited by theory, the inventors believe that as bone density decreases, the interior of the bone becomes negatively charged. Nanoparticles can enter the demineralized bone. If the nanoparticles are positively charged, they may be targeted to de-mineralized bone as a result of electrostatic attraction, thereby increasing either the delivery or retention, or both, of elements in the nanoparticle. Once on or inside the bone, the starch of the biopolymers is consumed or otherwise degrades and one or more elements and/or minerals released by the nanoparticle can help restore demineralized area.

Starch nanoparticles can also be targeted by way of size and/or charge selection towards a tumor. Once in the area of the tumor, the nanoparticles may attach to and/or be taken in by TAMs, or may degrade in the acidic environment of the tumor. Alternatively or additionally, starch nanoparticles can be targeted by one or more ligands to TAMs. For example, Harnessing Functionalized Polysaccharides for Medical and Dental Applications, a dissertation by Nathan Jones submitted to the University of Michigan in 2017 (ORCID iD: 0000-0002-5386-246X) and incorporated herein by reference, describes alpha-mannose functionalized polymer brushes for the of M2-polarized tumor associated macrophages. The sugar based targeting compounds described therein, or other variations such as mannan, etc., when mixed with starch in the method described in this application, become crosslinked to the starch nanoparticles thereby providing an actively targeted nanoparticle. In another example, International Publication Number WO 2013/081720, Aptamer Bioconjugate Drug Delivery Device, which is incorporated herein, describes methods of attaching aptamers to starch nanoparticles. Aptamers can be used to target a nanoparticle to TAMs. For example, aptamers that block the human IL-4 receptor alpha (IL4R(alpha) or CD124) were described in Roth et al. Aptamer-mediated Blockade of IL4R(alpha) Triggers Apoptosis of MDSCs and Limits Tumor Progression, Cancer Res; 72(6); 1373-83 (2012).

In a method described herein, biopolymer, i.e. starch, nanoparticles are made using an emulsion process. In brief, one or more biopolymers are dispersed or dissolved in water, the water is then dispersed (i.e. emulsified) in another phase, for example an oil phase, and the biopolymer is crosslinked while in dispersed droplets of the water phase in the dispersion or emulsion. The use of an oil as a second phase is optional but helps to load water-soluble reactants into the droplets of the water phase. However, another non-solvent of starch, for example ethanol or hexane, or a multi-phase aqueous system, may be used. The crosslinker may be a phosphate or polyphosphate crosslinker such as sodium trimetaphosphate (STMP) or sodium tripolyphosphate (STTP).

Optionally, the process may be a phase inversion emulsion (PIE) process. A schematic of a phase inversion process is shown in the Figure. In the example illustrated, a starch-based nanoparticle is made with a sodium trimetaphosphate (STMP) crosslinker. Initially an oil-in-water emulsion is formed which, after an increase in temperature, becomes a water-in-oil emulsion. A surfactant may be used to assist in the oil-in-water to water-in-oil transition and to select the temperature at which this transition occurs. The STMP is added so that the crosslinking reaction occurs within water droplets of the water-in-oil emulsion. Additional elements may be added to the water phase by adding them at any of the three stages shown in the Figure (separate water and oil phases, oil in water emulsion, water in oil emulsion).

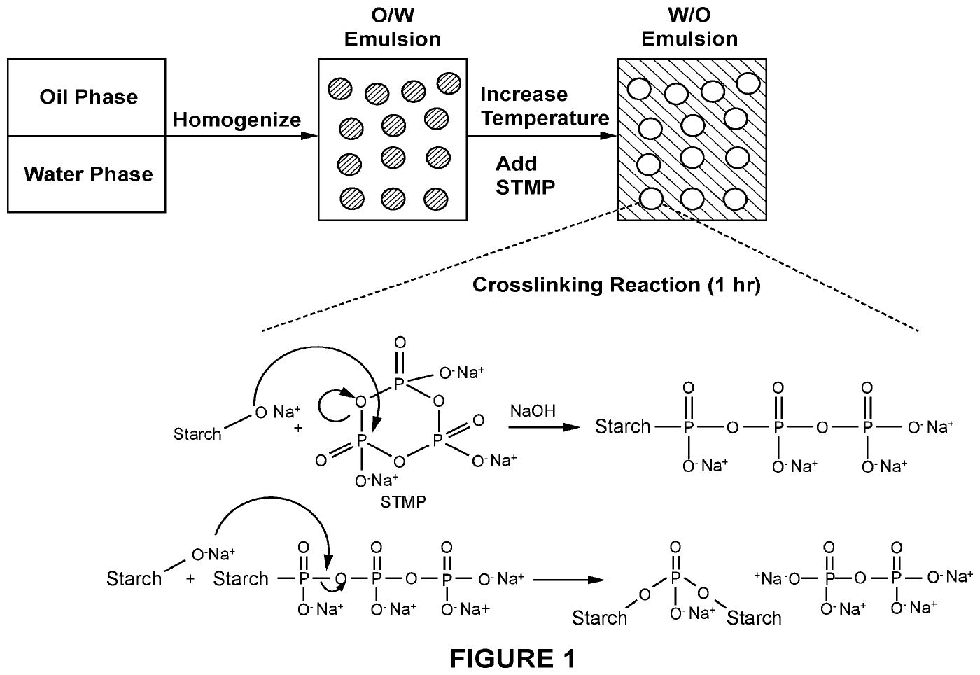

Referring to FIG. 1, an oil phase is homogenized with a water phase containing dissolved starch or dispersed starch nanoparticles. The oil may be, for example, paraffin oil or a food grade mineral oil. Alternatively, other food grade oils such as sunflower oil or olive oil may be used. After forming an oil-in-water emulsion, the temperature is increased to more than the phase inversion temperature (PIT) for the reaction conditions. The PIT may vary depending on the ratio of water to oil, the presence and type of any surfactants (for example Tween 85), the presence and type of any catalysts (for example NaCl) and the type of oil. In some cases, the PIT may be in the range of 25-60° C. Optionally, heating can be provided by the high shear mixer itself, for example by increasing the mixer speed to heat the mixture. As the water in oil emulsion is being heated or after the phase inversion is complete, the crosslinker is added. The reaction may then continue, for example for about an hour.

The biopolymer is crosslinked using a phosphate crosslinker such as STMP, typically under alkaline conditions. While other crosslinkers might be used, STMP is advantageously available in food grade preparations. The crosslinker provides a source of phosphorus, an element that may be useful for restoring demineralized bone. Part of the crosslinker (the inventors believe the part to be about 10-50% or 10-30%) reacts to form internal non-reversible (i.e. covalently bonded) crosslinks within the nanoparticles by way of monophosphate linkage. However, in addition to distarch monophosphate, side reactions may form other compounds such as monostarch triphosphate, monostarch monophosphate. The reaction is somewhat inefficient but dangling phosphate groups in either inorganic or organic compounds produced in the reaction or side reactions are available to form a strong associative complex with calcium and/or bisphosphonate either within the particle or later when deployed in bone. As mentioned above, a sugar (i.e. mannose) based targeting agent may also be added with the starch and crosslinked to the starch by way of the STMP.

In some examples, NaCl salt is used to provide high ionic strength in the water phase, which favors the STMP reaction to occur in a subsequent step. However, in other examples described herein, a bisphosphonate salt is used in place of, or in combination with, NaCl and to also provide bisphosphonate in the nanoparticle. For example, alendronate (sodium hydrate) may be added. The bisphosphonate can be added, for example, in the water phase produced prior to homogenizing to form the O/W emulsion in the Figure. Alternatively, the bisphosphonate can also be added while homogenizing to form the O/W emulsion or after formation of the O/W emulsion or, though with a possible decrease in bisphosphonate release time, after nanoparticle formation. In some examples, a calcium salt such as calcium chloride is added in the water phase. Optionally, the calcium salt, dry or in aqueous solution, is added into the W/O emulsion of the Figure after the STMP is added or, possibly with some decrease in calcium and/or bisphosphonate release time, after nanoparticle formation. Water soluble components are driven into the water droplets and at least partially react or otherwise associate with the nanoparticles. In another option, a calcium salt such as calcium chloride can be added in the water phase produced prior to homogenizing to form the O/W emulsion in the Figure either in place of bisphosphonate and/or NaCl or in addition to bisphosphonate and/or NaCl. STMP produces negative charges in the resulting nanoparticle. The addition of calcium can block some of these charges. However, an additional step such as cationization of the starch may be used to produce nanoparticles that are optionally positively charged (if desired) at neutral pH, or only at an acidic pH (i.e. 5.5 or less) that may be found within or near a demineralized bone.

The bisphosphonate and/or calcium are present with the phosphorous and biopolymer in a dispersion of small water droplets in an emulsion, for example a water-in-oil emulsion, optionally stabilized by surfactant. Each droplet containing biopolymer produces a crosslinked particle. Optionally, an emulsion of water in another phase may be used.

The emulsion or emulsions are preferably produced using an ultra-high shear mixer, for example a Silverson dissolver agitator. This mixer advantageously produces minimal air encapsulation and provides sufficient shear to produce nanoparticles averaging under 700 nm or under 500 nm in diameter or smaller. The starch may be cooked, chemically degraded and/or thermo-mechanically processed to help produce a solution or dispersion of starch in the water phase. Alternatively, smaller starch nanoparticles (20-200 nm) such as those produced by EcoSynthetix Inc. under the trademark EcoSphere™ can be used as the starch feed source. The resultant nanoparticles may have a mean or average size, measured for example by the peak in a dynamic light scattering (DLS) plot, the Z-average size (or harmonic intensity averaged particle diameter as described in ISO 13321 or ISO 22412) of a DLS measurement, or the mean or D50 value in a nanoparticle tracking analysis (NTA) measurement, of less than 1000 nm, for example in the range of 20-700 nm or 20-500 nm, or 20-300 nm. After breaking the emulsion, the water phase can optionally be centrifuged, for example at 4000 rpm for 1 minute, to separate the nanoparticles in the supernatant from unassociated precipitates in the pellet. The nanoparticles are optionally washed to remove traces of oil although if a suitable, i.e. food-grade, oil is used it is not necessary to completely remove all traces of oil. The supernatant can be freeze dried to obtain dry nanoparticles. The nanoparticles can be stored dry or, for a more limited time, in an aqueous dispersion, gel or paste.

The amount of cross-linker used may be 1 mol % to 50 mol % of STMP based on anhydrous glucose repeating units (AGU). Optionally, nanoparticles can be produced with 3 mol % to 50 mol % STMP, from 10 mol % to 50 mol % STMP, from 10 mol % to 30 mol % STMP, or for example about 30 mol % STMP. Particle size does not appear to be clearly related to the amount of STMP except that, in some examples, very low amounts of STMP (i.e. 1%) produced small nanoparticles (about 100 nm), low amounts of STMP (i.e. 1-5%) produced large nanoparticles (average size of about 300-500 nm) while larger amounts of STMP (5% to 50%) produced intermediate nanoparticles (about 100-300 nm). Without intending to be limited by theory, it is possible that samples made with very low STMP (i.e. 1%) do not incorporate substantially all of the available starch into nanoparticles although 3% STMP seems to be sufficient. Once sufficient crosslinker is available, the smaller size with larger amounts of STMP may be due to higher crosslinking and less swelling (as predicted by the Stokes-Einstein equation related to volume swell ratio) since the particles are hydrogels and their size is measured in a swollen state. It is also possible that particle size is influenced more by the amount of shear energy applied or other factors that could affect droplet size of the water in oil emulsion. In some cases, nanoparticles made with added calcium had zeta potentials near neutral, for example in a range from −5 to +5 mV at a pH of 7.0. Optionally, precipitates produced in the water phase that are not associated with the nanoparticles can be separated by centrifugation. The nanoparticles tend to remain in the supernatant of the centrifuged sample. The nanoparticles exhibit swelling behavior and appear to be hydrogels. For example, the nanoparticles retain water, but the amount of water retained by the nanoparticles decreases with increasing ion concentration.

The nanoparticles become more negatively charged (as measured by zeta potential) with increasing pH and STMP content. In some examples, the zeta potential of nanoparticles with 1-50 mol % AGU of STMP, without calcium salt added and without starch cationization, ranged from 0 to −65 mV across a range of pH and STMP content, or −10 to −22 mV at neutral pH. For example, samples made with 30% STMP, without calcium salt added and without starch cationization, were measured as having a zeta potential of −15 mV at a pH of 3, −45 mV at pH of 8, and further decreasing to −70 mV at pH of 12.

Adding calcium, for example as CaCl₂, but still without starch cationization (as described in more detail below) reduces the negative zeta potential of the nanoparticles. At near neutral pH and a 5% STMP content, the charge of the nanoparticles with calcium added can be in the range of −5 mV to 0 mV. Nanoparticles made with calcium and 30% STMP have a zeta potential in the range of −30 mV to −25 mV near neutral pH and without starch cationization. Without intending to be limited by theory, the added calcium may be capping the phosphate groups provided by the STMP. Optionally, the starch may be cationized to produce a further decrease in negative zeta potential, or to produce a positive zeta potential over a desired range of pH.

As an alternative to STMP, sodium tripolyphosphate (STTP) may be used as the crosslinker.

Optionally, the nanoparticles can be cationized, for example by the method described in International Publication Number WO 2017/070578, Detection and Treatment of Caries and Microcavities with Nanoparticles. Optionally, the starch may be cationized while in the water in oil emulsion. For example, glycidyl trimethyl ammonium chloride (GTAC), optionally with or pre-mixed with water and isopropyl alcohol or 2-proponol, may be added to the water phase before or after forming the water in oil emulsion. Alternatively, the starch may be cationized after the nanoparticles are formed. Alternatively, the starch may be cationized before the nanoparticles are formed, although in this case the starch is preferably first cooked or regenerated so that the cationization is not limited to the surface of the starch granules.

By selecting the amount of STMP, calcium if any and GTAC if any, the zeta potential of the nanoparticles may be made to be positive or negative at neutral pH or at an acidic pH (for example about 5.5). The choice of zeta potential may depend on the intended treatment and application method. For example, a negative zeta potential while in the bloodstream may assist with circulation of the nanoparticles by inhibiting bonding to blood proteins. However, a cationic zeta potential, or a nearly neutral zeta potential, may assist with targeting the nanoparticles to bone.

The nanoparticles may have a size of up to 2500 nm but preferably have a size of 1000 nm or less. The term “nanoparticles” as used herein is not limited to particles having a size of 100 nm or less as in the IUPAC definition but also includes larger particles, for example particles up to 2500 nm, or up to 1000 nm, for example in their largest dimension or in the diameter of a sphere of equivalent volume. Optionally, the nanoparticles may have a mean or average size as determined by peak intensity of a DLS plot, the z-average of a DLS measurement or the mean or D50 of an NTA measurement, in the range of about 100 nm to about 700 nm, about 100 nm to about 600 nm, or in the range of about 100 nm to about 500 nm, or in the range of about 200 nm to about 500 nm, or in the range of about 100 nm to about 400 nm. As mentioned above, particles in these size ranges will be called nanoparticles, which is consistent with common usage of that word in North America particularly for particles less than 1000 nm in size. However, in other parts of the world, and according to IUPAC definition, particles larger than 100 nm in size may alternatively be called microparticles.

Biopolymers, for example polysaccharides and proteins, and in principal any other biopolymer, and mixtures thereof, may be the biopolymer used in these processes. Any starch, for example waxy or dent corn starch, potato starch, tapioca starch, dextrin, dextran, starch ester, starch ether, carboxymethyl starch (CMS), and in principle any other starch or starch derivative, including cationic or anionic starch, and mixtures thereof, may be the biopolymer used in these processes. Any polysaccharide, cellulosic polymer or cellulose derivative, for example microcrystalline cellulose, carboxymethyl cellulose (CMC), any nanofibrillar cellulose (CNF), nanocrystalline cellulose (CNC), or cellulose ester, cellulose ether, and in principle any other polysaccharide, cellulose or cellulose derivative, and mixtures thereof, may be the biopolymer used in these processes. Proteins, for example zein (corn protein), casein (milk) or soy protein, and in principle any other protein or modified protein, and mixtures thereof, may be the biopolymer used in these processes.

Optionally, the nanoparticles may be prepared by a phase inversion emulsion process as described in U.S. Pat. No. 6,755,915, Method for the Preparation of Starch Particles. In this method starch particles are prepared in a two-phase system comprising steps of a) preparation of a first phase comprising a dispersion of starch in water, b) preparation of a dispersion or emulsion of the first phase in a second liquid phase, c) crosslinking of the starch present in the first phase, d) separating the starch particles thus formed. In some examples the second phase consists of a hydrophobic liquid and step b) consists in forming an oil-in-water emulsion. In some examples the second phase consists of a water-miscible non-solvent for starch.

The nanoparticles are stable in dry form. If stored wet, the nanoparticles may be kept in a closed container, for example as a sterile 5% w/w aqueous dispersion.

The nanoparticles can be combined with one or more supplemental carriers (i.e. water, excipients or extenders etc.) that are toxicologically and functionally acceptable to create a composition that can be administered to a person, for example orally, by injection at a tumor site, or by intravenous injection.

Other two-phase emulsions, for example water and alcohol or hexane, might be used. However, the oil phase helps achieve a high loading of non-oil soluble active agents in the nanoparticle. The oil may be a food grade mineral oil, or other, preferably food grade, oils such as sunflower oil or olive oil. A surfactant, for example Tween 85, is also used. The transition temperature may vary depending on the water to oil ratio, the type of oil, and the type and amount of surfactant.

International Publication Number WO 2017/070578, Detection and Treatment of Caries and Microcavities with Nanoparticles, is incorporated by reference. International Publication Number WO 2013/081720 A1, Aptamer Bioconjugate Drug Delivery Agent, is incorporated herein by reference. All of the patent publications and other publications mentioned herein are incorporated by reference.

The term “preferable” or variants thereof indicates that something is preferred but optional. Words such as “may” or “might” are meant to include the possibility that a thing might, or might not, be present.

The following example is provided to illustrate an embodiment and to provide further enabling disclosure but are not intended to limit any claimed invention.

### Example 1—Preparation of Starch Nanoparticles with 10% by Mass of Alendronate

In a 1 L plastic beaker 14.3 g of native waxy corn starch was dispersed into 400 g water. 3.1 g of 50% NaOH, 34.9 g of Tween 80 and 499.2 g of mineral oil were added. The dispersion was mixed using a Silverson ultra-high shear dissolver agitator starting at 4500 but with rpm increasing to 8850. An oil-in-water emulsion formed but converted to a water-in-oil immersion as the temperature reached about 40 degrees C. About 10 minutes later, temperature had increased further to about 67 degrees C. and the mixer speed was reduced to 7800 rpm. 13.8 g of NaCl and 2.4 g of Alendronate (alendronate sodium hydrate) were added. About 5 minutes later, mixer speed was reduced to 7400 rpm and 10.3 g of STMP was added. Mixing continued for another 75 minutes at a temperature of about 70 degrees C. Mixing speed was then reduced to about 1900 rpm to allow the mixture to cool. The pH was adjusted from about 11 to about 7.8 to 9.7 (for different samples) using HCl. After neutralizing the suspension was broken.

The nanoparticles had a Z-average diameter as determined by dynamic light scattering of about 550 nm. Mean diameter determined by nanoparticle tracking analysis was about 200 nm. The zeta potential of the particles was about −48 mV.

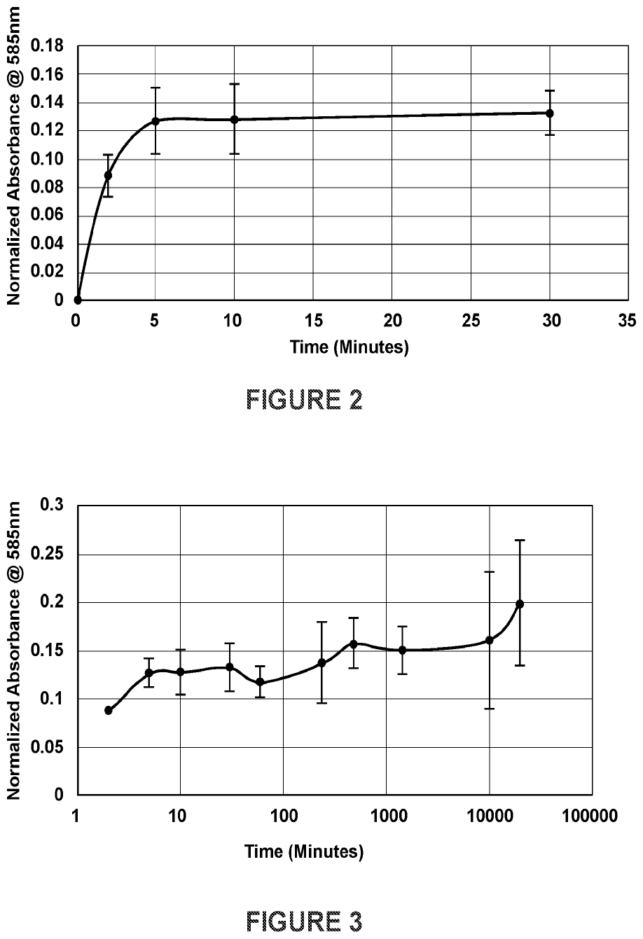

A colorimetric assay was used to confirm that the Alendronate had been incorporated into the nanoparticles and determine its release profile. Nanoparticles were put into dialysis tubes in Phosphate-buffered saline (PBS). Aliquots of dialysate were collected at various times and reacted with Ninhydrin. Ninhydrin reacts with the primary amine present on the Alendronate and changes to a purple color. Absorbance of the aliquots at 585 nm is related to the amount of Alendronate released from the nanoparticles. Measurements of absorbance measurements on aliquots taken at various times are given in FIGS. 2 and 3. As indicated in those figures, an initial burst of Alendronate was released in about 5 minutes. Thereafter, Alendronate continued to be released gradually over 2 weeks, which was the end of the trial. It is estimated that about 30% of the Alendronate added into the reaction had been released at the end of the trial.

1. A method of making nanoparticles comprising the steps of,
preparing a first phase comprising a solution or dispersion of starch in water;
preparing a dispersion or emulsion of the first phase in a second liquid phase such as an oil phase;
adding a bisphosphonate active agent to the first phase; and,
crosslinking the starch in the first phase with a phosphate crosslinker.

2. The method of claim 1 wherein the bisphosphonate active agent is added to the first phase before preparing an emulsion or dispersion of the first phase in the second liquid phase.

3. The method of claim 1 further comprising adding one or more multi-valent cations or one or more starch cationizing agents to the first phase.

4. The method of claim 1 wherein the one or more multivalent cations comprises calcium.

5. The method of claim 1 comprising adding one or more starch cationizing agents.

6. The method of claim 1 wherein the crosslinker comprises sodium trimetaphosphate, optionally added at between 3 and 50 mol %.

7. The method of claim 1 comprising mixing the emulsion of the water phase in an oil phase with sufficient shear to produce nanoparticles having an average or mean size in the range of 20-700 nm as determined by the Z-average size in dynamic light scattering (DLS) or as determined by the mean size in nanoparticle tracking analysis (NTA).

8. The method of claim 1 comprising incorporated the nanoparticles into an aqueous dispersion suitable for intravenous or tumor injection or to be taken orally, or into a pill.

9. Nanoparticles comprising starch, bisphosphonate and phosphorous.

10. The nanoparticles of claim 9 comprising calcium.

11. The nanoparticles of claim 9 comprising Alendronate or a sodium salt of a bisphosphonate active agent.

12. The nanoparticles of claim 9 with a targeting ligand.

13. The nanoparticles of claim 9 incorporated into an aqueous dispersion suitable for intravenous or tumor injection or to be taken orally, or incorporated into a pill

14. (canceled)

15. A method of treating osteoporosis, a skeletal condition or cancer comprising administering the nanoparticles of claim 9 to a patient.

16. The nanoparticles of claim 8 wherein the phosphorous is present in starch-phosphate compounds and/or dangling phosphates.

17. The nanoparticles of claim 12 wherein the targeting ligand is a TAM targeting ligand.

18. The nanoparticles of claim 12 wherein the targeting ligand comprises mannose or an aptamer.

19. The method of claim 4 wherein the calcium is added to the first phase as a calcium salt.

20. The method of claim 4 wherein the calcium is added to the first phase while or after preparing the emulsion or dispersion of the first phase in the second liquid phase.

21. The method of claim 5 wherein the one or more starch cationizing agents are in an amount sufficient to produce nanoparticles having a positive zeta potential at a pH of 5.5 or less or at a pH of 7.0 or less.
